(19) United States
(12) Patent Application Publication
(10) Pub. No.: US 2019/0274758 A1
(43) Pub. Date: Sep. 12, 2019

(54) Apparatus and method for the treatment of Epidermal Dysplasias

(71) Applicant: Emblation Limited, Alloa (GB)

(72) Inventors: Gary Beale, Alloa (GB); Eamon McErlean, Alloa (GB); Matthew Kidd, Alloa (GB)

(21) Appl. No.: 16/277,146

(22) Filed: Feb. 15, 2019

Related U.S. Application Data
(60) Provisional application No. 62/710,314, filed on Feb. 16, 2018.

(51) Int. Cl. A61B 18/18; A61K 9/00

(52) U.S. Cl. CPC A61B 18/1815; A61B 2018/00452; A61K 9/0014

(57) ABSTRACT

Described herein are methods and apparatus for treating or preventing epidermal dysplasias, including, for example, dysplastic epidermal lesions and dermatological pre-cancerous disease.

### CROSS REFERENCE TO RELATED APPLICATION

This application claims the benefit of priority under 35 U.S.C. § 119(e) to U.S. Provisional Patent Application Ser. No. 62/710,314 filed Feb. 16, 2018, which is incorporated herein by reference.

### FIELD OF THE INVENTION

The present invention provides methods and apparatus for treating or preventing epidermal dysplasias, including, for example, dysplastic epidermal lesions, dermatological pre-cancerous disease.

### BACKGROUND

Epidermal dysplasias are cellular abnormalities present in the epidermal layer of skin of a human. The abnormalities relate to deviations from normal development, growth and differentiation all of which have the potential for malignant transformation. The most common condition in this group of dermatological conditions is actinic (solar) keratosis but also includes actinic cheilitis, arsenical keratoses, and PUVA keratosis. As the main cell types in the epidermis are keratinocytes, the term keratosis is associated with the conditions in this area.

### Actinic Keratosis

Actinic keratosis (AK) are also known as solar keratosis due to the origin of their formation being driven by over-exposure of skin to the sun. The sun emits a wide spectrum of radiation of which the ultra-violet region is sub divided into 3 wavelength ranges: UV-A at 315 to 400 nm, UV-B at 280 to 315 nm and UVC at 100 to 280 nm. The upper atmosphere of earth absorbs most of the UVC range with the lower, ozone layer, absorbing 90% of UV-B and less than 50% of UV-A. As the ozone layer is thinner around the tropics regions of the earth and where there are notable holes, the impact on humans is more noticeable e.g. in Australia, AKs are found in 40-60% of people aged 40 years, far higher than those in the northern latitudes. Equally, those who use sunbeds excessively will also experience greater risks of AK. Also as a consequence of sun exposure at the root cause of AKs, it is the face, ear, scalp, hand, and forearm sites that are of greatest susceptibility. Transplant recipients are an example of those patients who are immunosuppressed or immunocompromised and will experience AK earlier in their life in comparison with healthier individuals. Genetic makeup also plays a role in risk with those of a fair complexion, who do not tan readily, more susceptible to developing the lesions.

Diagnostic biopsy is undertaken in only a small percentage of AKs diagnosed clinically as the clinical accuracy of recognition is up to 90%. Typically, they are presented as circumscribed scaly erythematous lesions all of similar appearance on the same patient and usually less than 10 mm in diameter. They feature a rough surface scale, usually white, although in patients with some skin types AK are often more easily felt than seen. Although termed as flat some lesions can have significant amounts of scale when the AK is hypertrophic in nature that is when the cells are enlarged rather than in high number (hyperplastic). The hyperplastic variant has a relatively high rate of malignant transformation. Non-variant AKs may remit, or remain unchanged for many years. Up to 20% may progress to squamous cell carcinoma if left untreated. This is similar to other neoplastic growths in the body that may take many years to fully transform such as cervical intraepithelial neoplasia (CIN), when the potentially premalignant transformation and abnormal growth of squamous cells on the surface of the cervix is allowed to form.

Abnormalities in DNA synthesis in keratinocytes in the skin around the lesion suggest that there is a gradual stepwise progression from sun-damaged epidermis to clinically obvious keratoses and then, eventually to squamous cell carcinoma. UV radiation is a complete carcinogen in that it both induces the initial genetic mutations in keratinocytes and promotes tumour cell expansion. UV-A penetrates the skin more deeply, produces reactive oxygen species (ROS), and causes oxidative damage in nucleic acids, membrane lipids, and proteins. Oxidative damage disrupts normal signal transduction pathways and cellular interactions, leading to abnormal proliferation. The mutagenic effects of UV-A are generally regarded to originate from oxidative damage to DNA. The higher-energy UV-B has long been recognised as a potent carcinogen because of its direct mutagenic effects on DNA. The basal layer of the epidermis is the site of dividing cells and is therefore more likely to give rise to a cancer cell after mutagenesis than are the non-dividing cells of the upper epidermis.

Mutations in TP53 are known to play a crucial role in the development of AK. [PMED 7997263] When exposed to UVB light, mice with severe combined immune deficiency (SCID) with transplanted human skin will develop actinic keratoses associated with specific TP53 mutations. [32] UVB light has also been shown to downregulate the tumour suppressor PTEN (phosphatase and tensin homologue deleted on chromosome 10) in actinic keratosis, via an ERK (extracellular signal-regulated kinase)/AKT-dependent mechanism.

A further influence on AK development can be the human papillomavirus (HPV). HPV is regarded as a co-carcinogen because the E6 and E7 viral proteins found in a broad spectrum of HPV types prevent apoptosis, independent of the status of p53. The apoptosis-resistant cells are then vulnerable to the accumulation of additional UV-induced genetic damage, contributing to unregulated cell proliferation. Secondly, the E6 protein of HPV5 and HPV8 can down-regulate the expression of IL-8, a prominently expressed cytokine induced upon UV exposure to keratinocytes thus weakening the repair response to UV-induced DNA damage.

The treatment of AKs may be driven by a patient's cosmetic desire or from a clinicians decision when they are growing rapidly, the patient is immunosuppressed or there is an elevated risk of cancer. In the latter case, surgical removal is common as the lesion can undergo lab analysis to diagnosis, this leaves the patient with a scar however. Other forms of treatment include freezing with liquid nitrogen (Cryotherapy). The efficacy of cryotherapy varies by freeze time from 39% for less than 5 seconds to 83% for periods greater than 20 seconds. However such aggressive therapy durations may cause depigmentation and scarring [PMC4065271]. Hypopigmentation occurs in 29% of cleared lesions, while hyperpigmentation may occur in up to 6% of cases. This is due to the susceptibility of melanocytes to freezing. A disadvantage of cryotherapy is that large areas or densely occurring AKs cannot be treated.

Non-surgical treatments for AKs based on creams and can cover entire sections of skin e.g. arms or forehead. In some cases a cream or gel can be prescribed for use at home. These may include 5-fluorouracil (5-FU), Imiquimod, Ingenol mebutate or gel diclofenac. The latter is helpful when applied to milder AKs as the other agents often cause temporary inflammation, redness and soreness of the treated areas.

5-FU causes cell death in proliferating cells by reducing DNA synthesis. At a typical concentration of 5% a 4 week course yields a 67% efficacy however, of these 54% may relapse after 12 months. Additionally, the patient's wider health must be taken into consideration as only 10% is absorbed in the skin with 80% of the remainder being processed in the liver where the toxicity can cause more significant side effects in organs.

Imiquimod is a topical medication based on a synthetic compound of the imidazoquinoline molecule. The compound acts on both the innate and adaptive immune systems. The main biologic effects of imiquimod are mediated through agonistic activity towards toll-like receptors (TLR) 7 and 8 located on the surface of antigen-presenting cells (APCs) such as dendritic cells, macrophages and Langerhans cells. Consequently, the activation numerous cytokines and chemokines, tumour necrosis factor (TNF) alpha, Interferon (IFN) gamma, that leads to transcription of NF-κB (nuclear factor kappa B). NF-κB is needed for proper innate and adaptive immune responses, dysregulation of NF-κB can lead to inflammatory diseases and tumorigenesis. As IL-12 and members of its family such as IL-27 are also upregulated with Imiquimod, the priming of profound T-helper (Th1) cell differentiation provides assistance to the adaptive arm of the immune response. Additionally, it induces E-selectin on tumour vessels and consequent infiltration by cutaneous lymphocyte-associated antigen-positive skin-homing cluster of differentiation (CD8+) cytotoxic T cells.

The application of the Imiquimod cream is commonly made in either 5% or 3.75% concentrations. The 3.75% preparation allows for a shorter duration of treatment over a larger skin surface area (200 cm2 vs. 25 cm2 for 5% cream) [PMID: 21137610]. Absolute clearance rate is higher for the 5% cream, at 45%, compared to 35% for daily use of the 3.75% preparation [PMID: 23345970]. If used three times per week rather than daily, the 5% cream can give up to 57% clearance at 16 weeks [PMID: 15996416].

Failure of maintaining the adherence of Imiquimod treatment regime by patients is one of the reasons it is not more effective. Side effects may also contribute to complete compliance, they include: dermal ulcer, burning sensation, desquamation, edema, excoriation, exfoliation of skin, pruritus, skin erosion, and erythema [PMC3580198].

The burden of self-applied creams is a problem faced by other topical cream applications such as Diclofenac, another common treatment for AK. Diclofenac is a nonsteroidal anti-inflammatory drug that reduces the production of prostaglandins through inhibition of cyclooxygenase 2 (COX-2). COX-2 is seen as an important mediator in the development and progression of AK [10.1016/j.jaad.2012.09.053] as it permits the formation of prostaglandin E2 (PGE2), which in turn enhances tumour proliferation, angiogenesis, and inflammation, and inhibits apoptosis. A typical regime for AK is Diclofenac 3% in hyaluronic acid 2.5%, applied twice daily for 60 to 90 days. Complete response rates for AK treated with diclofenac vary considerably, with efficacy ranging from 29% [PMID: 9431711] to 81% [PMID: 9382562] depending on the study. The side effects include skin rash (nettle rash); breathing difficulties (wheezing); runny nose (allergic rhinitis), itching, rash, skin redness, inflammation, contact dermatitis, pain and blistering [Solaraze™ package insert PL No: 08929/0341]. Similar to other NSAIDs [fda 21005 lbl] caution must be used when used on patients with a history of stomach ulcer, bleeding from the stomach, heart, liver and kidney problems which are very common conditions in the population that experience AK.

The side effects of Ingenol mebutate (a natural extract of Euphorbia peplus) are somewhat milder with redness, flaking or scaling, crusting, or swelling common but short lived due to the concentration and treatment duration. Topical ingenol mebutate gel is approved for the treatment of AK in 2 concentrations: 0.015% applied once daily for 3 days for lesions on the head and 0.05% applied once daily for just 2 days for lesions on the trunk [10.1002/14651858.CD004415.pub2]. The exact mechanism of action of ingenol mebutate is unknown, but it is able to induce rapid and direct cell death through immediate cytotoxicity and a neutrophil-dependent inflammatory response [PMH0064601]. Ingenol mebutate has resulted in complete response rates of 42% for lesions on the face and neck and 34% for lesions on the trunk and extremities [10.1056/NEJMoa1111170]. The low efficacy may be related to the cytotoxic effects of ingenol mebutate and the response of the immune system characterised by T-cell deficiency, polymorphic neutrophil deficiency, and other factors such as intracellular calcium levels [10.1016/j.adengl.2016.08.016].

A combination therapy that uses a topical cream in conjunction with a special light shone onto the affected areas is called Photodynamic Therapy (PDT). Delta-aminolevulinic acid is such a cream compound and is a component of the heme biosynthetic pathway that accumulates preferentially in the dysplastic cells inherent in AK lesions. Once inside these cells, it is enzymatically converted to protoporphyrin IX, a potent photosensitizer. With exposure to light of an appropriate wavelength, oxygen free radicals are generated and cell death results [https://reference.medscape.com/medline/abstract/9586814]. Patients experience pain, similar in scope to the pain resulting from topical 5-FU, in the areas treated. The treated lesions may become erythematous and crusted. Compared with other destructive therapeutic options such as cryotherapy, PDT may offer better cosmetic results and higher patient preference compared to surgical removal. The efficacy of PDT varies depending on the variables of activating cream or gel and the wavelength or duration of light energy; one study claims the equivalence with surgery [www.nice.org.uk/guidance/ipg155]. The adverse events in the trial were 52% and burning was cited in 31% of patients which, combined with the hospital delivery of the therapy means it is a challenge to expand the service to the large population in need of the treatment.

In local hyperthermia, heat is applied to a small area, such as a tumour, using various techniques that deliver energy to heat the tumour. Different types of energy may be used to apply heat, including laser, radiofrequency, and ultrasound. Microwave radiation can heat deeper layers resulting in a superior deposition of energy within the diseased region. Additionally, microwave energy causes heating and gradual desiccation of tissue without generating the harmful smoke plume associated with high energy vaporization.

It is established that heat shock proteins (HSP) are produced in response to various tissue stresses or damage resulting from physical or environmental influences. Heat shock proteins are a class of functionally related proteins whose expression is increased when cells are exposed to elevated temperatures or other stress. It has been suggested that the heat shock proteins may protect the cells from other stressors or against further damage. Heat shock proteins are also involved in antigen presentation, steroid receptor function, intracellular trafficking, nuclear receptor binding, and apoptosis. Typically exposure of cells to a heat shock temperature of 42 degrees C. results in transient activation of heat shock factor (HSF). The DNA-binding activity increases, plateaus, and dissipates, during which the intracellular levels of heat shock protein increase. Heat shock proteins can perform specific functions, for example extracellular and membrane-bound heat-shock proteins, especially HSP70 are involved in binding antigens and presenting them to the immune system.

The upregulation of the heat shock proteins is a principle part of the heat shock response and is primarily induced by the heat shock factor. Cellular stresses, such as increased temperature, can cause proteins in the cell to denature. Heat shock proteins bind to the denatured proteins and dissociate from HSF to form trimers and translocate to the cell nucleus to activate transcription resulting in the production of new heat shock proteins which bind to more denatured cells.

In research into the physiological heating effects of electromagnetic fields, high-frequency microwave energy (existing between 500 MHz to 200 GHz) has been reported to thermally produce elevated levels of specific heat shock proteins in tissue for example Ogura, British Journal of Sports Medicine 41, 453-455. (2007)) teaches that HSP90, HSP72, HSP27 levels are significantly higher in heated vastus lateralis muscle compared with unheated controls. Tonomura et al. (J Orthop Res. 26(1):34-41. (2008)) teach that in vivo HSP70 expression in rabbit cartilage increases with the application of moderate levels of microwave power (20-40 W). Additionally, de Pomerai et al. (Enzyme and Microbial Technology 30, 73-79 (2002)) teaches that prolonged exposure to weak microwave fields (750-1000 MHz, 0.5 W) at 25° C. induces a heat-shock response in transgenic C. elegans strains carrying HSP16 reporter genes.

In other unrelated research Shevtsov M and Multhoff G (2016) Heat Shock Protein—Peptide and HSP-Based Immunotherapies for the Treatment of Cancer Front. Immunol. 7:171 (10.3389/fimmu.2016.00171) teaches that tumours can be treated using vaccines containing heat shock proteins as immunologic adjuvants (HSPs). Amongst other pathways, the systemic antitumor immune response was found to be mediated by CD4+ and CD8+ T cells. It has been speculated that HSPs may also be involved in binding with protein fragments from dead malignant cells and highlighting them to the immune system thus boosting the effectiveness of the vaccine, e.g Oncophage (Antigenics Inc, Lexington, Mass.).

Heat shock proteins not only carry antigens but can also induce maturation of dendritic cells, resulting in a more efficient antigen presentation. It is known that hyperthermia can promote the activation of the Langerhans cells. Langerhans cells are the dendritic cells of the skin which continuously monitor the extracellular matrix of the skin and capture, uptake and process antigens to become antigen presenting cells (APCs). Particles and antigens are carried to draining lymph nodes for presentation to T lymphocytes. T cells release chemokines which cause the skin to be infiltrated by neutrophils, resulting in a swelling response which has been observed by Gao et al. (Chin Med J (Engl), 122(17):2061-3 (2009)) to occur before resolution of a HPV infection which is particularly relevant to HPV-positive melanoma states.

The application of mild localised hyperthermia of a pulsed nature may, for example, inhibit PI3K helping the immune system in identifying the underlying tumour or lesion, promoting Langerhans cells and heat shock proteins to identify and present tumour/lesion fragments to the immune system causing a localised inflammatory response followed by eradication.

The emerging understanding is that hyperthermia treatments work at multiple levels via complex complementary mechanisms involving a variety of signalling and trafficking molecules.

### SUMMARY

It is proposed here that microwave energy may be employed as a double-action (heat shock promoting, immunostimulatory) treatment for epidermal dysplasias.

The term “epidermal dysplasias” may embrace diseases and/or conditions which are characterised by cellular abnormalities present in the epidermal layer of skin of a human. Generally speaking, epidermal dysplasias include diseases, syndromes or conditions in which the cells (for example the keratinocytes), tissues and structures of the epidermal layers of the skin exhibit aberrant (or deviant) development, growth and/or differentiation. Thus, examples of “epidermal dysplasias” may be referred to as “epidermal dysplastic skin lesions”. One key feature of these dysplasias (or lesions) is that they have the potential for malignant transformation. The term “epidermal dysplasias” may include dermatological pre-cancerous disease and the specific dermatological conditions referred to as actinic (solar) keratosis, actinic cheilitis, arsenical keratoses and PUVA keratosis.

Hereinafter, the term “dysplastic skin lesion” (or “lesion”) will be used to embrace all forms of “epidermal dysplasia”. Further, it should be noted that a “dysplastic skin lesion” may comprise “dysplastic cells”.

The microwave treatment mechanism may thermally instigate the production of heat shock factor HSF thus elevating the level of heat shock proteins in and proximal to a dysplastic skin lesion as a means to invoke an immune response by associating the lesion with the elevated heat shock proteins. This localised thermal increase can be achieved using a precise deposition of energy at the location of lesions which is readily achievable using microwave energy. As in the case of dysplastic skin lesions, this energy can be delivered locally in precise locations of, on or within the body; this overcomes the systemic side effects with some cytotoxic compounds that progress throughout the body after initial penetration at the skin interface.

Localised microwave hyperthermia can be used to raise the temperature at the site of the tissue containing the dysplastic skin lesion thus promoting the immune response specific to the cells in the volume.

Microwave energy is also suspected of having immunostimulatory properties and as such may also be used as a means to stimulate or enhance a host immune response. That immune response may be local to the area targeted by or exposed to the microwave energy. This phenomenon is discussed in more detail below but the stimulated immune response may be sufficient to facilitate the clearance and/or resolution of an epidermal dysplastic skin lesion. Note, the microwave energy based methods of this disclosure may not be “ablative”—that is to say, they may not cause tissue destruction or necrosis. In some cases, the microwave energy may be used at an immunostimulatory dose (and not at a tissue ablative or necrotic dose).

Typically the dielectric properties of materials are measured relative to those of air and referred to as epsilon relative to air (Er) where air is Er=1. Dielectric properties have been researched by many authors (C. Gabriel, S. Gabriel and E. Corthout: The dielectric properties of biological tissues: I. Literature survey, Phys. Med. Biol. 41 (1996), 2231-2249) and it is known that the dielectric properties of diseased tissue (for example, tissue identified as exhibiting symptoms consistent with epidermal dysplasia) may differ when compared with normal tissue. The effect of electrical conductivity is the second factor that dictates the heating effects of microwaves in tissue. Thus, applicators that electrically match with the range of epsilon relative values and conductivity may be used to ensure energy is efficiently delivered into, for example, tissues with epidermal dysplasia.

In biological tissues, the primary influence on conductivity is the water content which is known to vary from normal skin to for example AK lesions. The stratum corneum hydration can be used as a surrogate measurement of AK thickness because hydration gradually decreases in keratotic tissue [DOI: 10.1038/srep33952].

In view of the above, the dielectric properties/constant of tissue associated with an epidermal dysplasia lesion, may be lower than that of normal (not diseased) tissue. As such, a tissue dielectric property/constant may form the basis of a procedure or method for the diagnosis or detection of an epidermal dysplasia lesion (the “dysplasia” altering the dielectric properties of the tissue). For example, tissue dielectric properties may be exploited in methods of diagnosing or detecting diseases and/or conditions characterised by or causing, changes in levels of keratin in tissue(s). Thus, a dielectric property or constant may be exploited in a method of diagnosing or detecting a disease, condition or syndrome associated with, comprising or caused or contributed to by an epidermal dysplasia lesion.

For example, the dielectric constant of a test tissue may be compared to a control dielectric constant, wherein if the dielectric constant of the test tissue is different than that of the control dielectric constant, the test tissue may be identified as transforming along the dysplastic spectrum.

A control dielectric constant may be that associated with a tissue which is not diseased or dysplastic (for example, tissue exhibiting one or more symptoms associated with Actinic keratosis (AK)). For example, where the tissue is AK tissue, in accordance with the information presented above, a fixed value control dielectric constant may exist within the range between Er 30 to Er 45.

A method of detection or diagnosis as described herein may be performed in vitro or in vivo. An in vitro method may be performed on a sample, for example a tissue biopsy.

In one application, a procedure or method for the diagnosis or detection of a disease or condition which alters the dielectric properties of a tissue (for example a dysplastic condition such as AK) may be conducted on a test tissue derived from a lesion suspected of having a dysplastic aetiology (for example from a tissue exhibiting one or more symptoms associated with AK or other some other lesion identified as being potentially of “epidermal dysplasia” aetiology) or a biopsy therefrom. Specifically, the dielectric properties of the tissue or sample may be compared to the corresponding control dielectric property from the same or a similar tissue, wherein if the dielectric property (constant) of the test tissue is lower than the corresponding control value, the test tissue may be diagnosed or identified as potentially comprising (or deriving from) an epidermal dysplasia (such as, for example AK).

A positive diagnosis (of an epidermal dysplasia or AK) may then lead to the use of any one of the microwave energy techniques described herein to resolve the relevant disease or condition.

As a penetration depth of only a few mm may be required a frequency in the range 2.45 GHz to 15 GHz is may be desirable as lower frequencies may penetrate too far, damaging deeper, healthy, tissues unnecessarily. The power level and energy density of application will also affect the depth of penetration, therefore, lower frequencies may be used with appropriately designed applicators and treatment profiles.

The present invention is based on the finding that microwave energy may be used to treat dysplastic disease states in epithelial volumes.

Thus disclosed herein is a method of treating, curtailing or preventing an epidermal dysplastic condition (including, for example conditions classified as AK or AK type), said method comprising administering a therapeutically effective amount or dose of microwave energy.

The method may be applied or administered to a human or animal subject and to any tissue or region thereof. The subject may be any subject having or suffering from any type or form of epidermal dysplastic condition (including AK) or to a human or animal subject predisposed or susceptible thereto.

The method may be applied or administered to any tissue susceptible to the development of an epidermal dysplastic condition, including, for example, the skin and/or mucosal tissues.

One of skill will appreciate that in order to resolve any type or form of dysplastic skin lesion described herein and/or to reduce the symptoms thereof, one or more treatments with microwave energy may be required.

Without wishing to be bound by theory, the inventors hypothesise that the induction of hyperthermia by targeted application of microwave energy induces the production of heat shock proteins in diseased or damaged tissue. This may lead to the activation of antigen presenting cells (such as dendritic/Langerhans cells) which process antigen (including host, microbial or other foreign antigen) for presentation to T cells. Furthermore, the induction of hyperthermia may inhibit the PI3K pathway in Langerhans cells facilitating the induction of a potent host immune response. Localised thermal increase can be achieved using a precise deposition of energy to a diseased tissue (for example a lesion), readily achievable using microwave energy.

Further, it has been noted that when used at energy levels which do not result in any substantial tissue damage or ablation, subjects may exhibit good clinical responses (Ivan Bristow, Wen Chean Lim, Alvin Lee, Daniel Holbrook, Natalia Savelyeva, Peter Thomson, Christopher Webb, Marta Polak, Michael R. Ardern-Jones. Microwave therapy for cutaneous human papilloma virus infection. European Journal of Dermatology. 2017; 27(5):511-518. doi:10.1684/ejd.2017.3086). Again, without wishing to be bound by theory, the microwave energy based treatments described herein have been shown to induce an immune response which facilitates the (at least partial) clearance and/or resolution of a dysplastic skin lesion. The immune response may be a partially or fully protective immune response. The immune response may be a local response—that is a response which “local” to the site of microwave energy treatment or the dysplastic skin lesion to be treated. The immune response may be a cell-based immune response involving the induction, proliferation and/or activation of one or more innate immune system mechanisms/pathways, immune system cells (T cells and the like) and/or cytokines. Thus, there is provided a method of stimulating an immune response in a subject, said method comprising administering a subject an amount or dose of microwave energy. Further, there is provided, microwave energy for use in a method of stimulating an immune response in a subject. As stated, the immune response may be a local response and/or a cell/cytokine based response.

The subject may be any subject diagnosed as suffering from a dysplastic skin lesion as described herein. Additionally, or alternatively, the subject may be susceptible or predisposed any of the dysplastic conditions (including dysplastic skin lesions) described herein.

Thus, in one application, a method of raising an immune response via the use of microwave energy may be applied to a subject suspected of suffering from a dysplastic skin lesion. In such circumstances, the method may involve applying microwave energy to dysplastic skin lesion so as to induce a local immune response. As stated, the raised immune response may then be sufficient to resolve the one or more dysplastic skin lesion(s).

The method of stimulating an immune response in a subject may exploit microwave energy applied at a level which does not result in any substantial macroscopic and/or histological changes in the tissue subjected to the microwave treatment. The microwave energy may be used at a level which is sub-apoptotic and/or which induces in human skin, mild macroscopic epidermal changes, microscopically minor architectural changes and slight elongation of keratinocytes without evidence of dermal collagen sclerosis. Higher levels of microwave energy may be used (for example 100J or >100 J) and this may result in more significant dermal and/or epidermal changes, including gross tissue contraction, spindled keratinocytes with linear nuclear architectural changes and subepidermal clefting. The microwave energy may be applied at a sub-clinical level, that is an energy level which might not in itself result in resolution or clearance of the dysplastic skin lesion affecting the targeted tissue.

The microwave energy may be applied at an energy of anywhere between about 1 J and about 500 J, for example, about 5 J to about 200 J. The microwave energy for use in a method of stimulating an immune response in a subject may be used at about 5 J, about 10 J, about 50 J, about 100 J or about 200 J.

The microwave energy for raising or stimulating an immune response may be applied for any suitable duration of time. The microwave energy may be applied for anywhere between about 0.1 s and about 1 minute. The microwave energy may be applied for about 1 s, about 5 s about 10 s, about 20 s or about 30 s. The microwave energy may be applied as multiple bursts or pulses of the same or different duration and/or of the same or different energy level. Each applied microwave energy burst/pulse may last for the same or a different duration. An applied amount of microwave energy may be described as a “microwave energy dose”.

A subject may be delivered one or more microwave energy doses over a predetermined period of time. For example, a subject may be administered a single dose on 1 day or multiple doses over the same day, each dose being separated by a non-dosing period. Additionally or alternatively, a subject may be administered other doses on subsequent days. A treatment (comprising one or more microwave energy doses) may last a day, multiple days or one or more weeks months or years.

Where the dysplastic skin lesion to be treated is small, for example less than about 7 mm in diameter, a single dose may be applied to a single site within or on the lesion. Where the lesion is larger, for example larger than about 7 mm, the lesion may be applied multiple doses in a manner that ensures that the entire surface of the lesion has been exposed to microwave energy.

The immune response stimulated or induced by the methods described herein may comprise the activation of certain cell types, increased antigen presentation and modulated cytokine production. More specifically, an immune response stimulated or induced by a microwave energy based method of this invention may comprise keratinocyte activation, enhanced signalling between keratinocytes and dendritic cells which in turn enhances the level of HPV antigen cross-presentation to CD8+ T lymphocytes and enhanced IL-6 synthesis from keratinocytes.

One of skill will note that IL-6 is a pro-inflammatory cytokine with an important role in anti-viral immunity and which has been shown to induce rapid effector function in CD8+ cells. As such (and without wishing to be bound by theory), the microwave energy based methods described herein, which methods induce IL-6 synthesis, may facilitate the induction of a cytokine based anti-viral immunity.

Additionally, it has been shown that the microwave energy based methods of this invention might modulate certain other aspects of the host immune response. For example, the microwave energy may modulate interferon regulatory factor (IRF) expression. IRFs have been shown to be central to the regulation of an immune response. For example, IRF4 is essential for cytotoxic CD8+ T cell differentiation and its up-regulation in dendritic cells has been shown to enhance CD4+ differentiation, thereby potentially enhancing both CD8+ immunity and T cell help following microwave treatment. IRF1 expression has also been implicated in the modulation of HPV infection. It has been found that IRF1 expression is down-regulated in response to the application of microwave energy and thus IRF1 may represent another therapeutic target for the treatment and/or prevention of an HPV infection.

Thus the microwave energy based immunostimulatory methods of this invention might be used to achieve any one or more of the following:

(i) modulation of immune cell activation;

(ii) modulation of antigen presentation;

(iii) modulation of cytokine expression; and/or

(iv) modulation of IFR

(v) modulation of ERK and PI3K pathway

(vi) modulation of IL6

(vii) modulation of IL12

(viii) modulation of T cells

As stated, the stimulated immune response may be effective to facilitate the resolution and/or clearance of a dysplastic skin lesion.

One of skill will appreciate that unlike with vaccines and checkpoint inhibitors which are specific to a particular pathway, species or phenotype of dysplastic cells, microwave energy may be used to treat dysplastic lesions irrespective of the pathway/species/phenotype/class. This is particularly important for dysplastic conditions which pass through phases of development where the proliferation mechanism changes thus their susceptibility to anti-tumour treatments can be hard to predict or match to available agents.

One of skill will appreciate that any method in which an immune response can be stimulated, enhanced or induced at the site of a dysplastic lesion, may represent an improvement over prior art methods which focus of the direct and individual treatment of each lesion. A method which stimulates, enhances or induces some form of local (cell and cytokine) based immune response at a single site of infection may facilitate the clearance of a number of similar lesions in the same area.

Microwave energy according to this invention may have a frequency of between about 500 MHz and about 200 GHz. In other embodiments, the frequency of the microwave energy may range from between about 900 MHz and about 100 GHz. In particular, the frequency of the microwave energy may range from about 5 GHz to about 15 GHz and in a specific embodiment has a frequency of 8 GHz.

It should be understood that the methods of treatment described herein may require the use of a microwave energy having a single frequency or microwave energy across a range of frequencies.

The invention further provides an apparatus for use in treating any of the epidermal dysplasia conditions (and/or associated syndromes or diseases) described herein, said apparatus comprising a microwave source for providing microwave energy and means for administering or delivering the microwave energy to a subject to be treated. The apparatus provided by this aspect of the invention may be used in any of the therapeutic methods described herein.

Advantageously, the microwave energy emitted or produced by the apparatus elevates or raises the temperature of the subject to be treated and/or stimulates a local immune response as described herein. In one embodiment, the microwave energy may cause targeted or localised hyperthermia in a tissue of the subject, including, for example, the skin and/or mucosal membrane. The temperature elevation may be localised to the surface of the skin and/or to the epidermal, dermal and/or sub-dermal layers thereof (including all minor layers that lie within).

The apparatus may further comprise means for controlling at least one property of the microwave energy produced by the microwave source. For example, the means may control or modulate the power, frequency, wavelength and/or amplitude of the microwave energy. The means for controlling the microwave energy may be integral with the apparatus or separately formed and connectable thereto.

In one embodiment, the microwave energy source may produce microwave energy at a single frequency and/or microwave energy across a range of frequencies. The means for controlling at least one property of the microwave energy may permit the user to select or set a particular microwave or microwaves to be produced by the apparatus and/or the properties of the microwave(s) produced.

The apparatus may further comprise means for monitoring the microwave energy produced or generated by the microwave source. For example, the apparatus may include a display indicating one or more properties of the microwave energy.

In one embodiment, administering or delivering the microwave energy to a subject to be treated may be achieved through the use of an applicator formed, adapted and/or configured to deliver or administer microwave energy to the subject. The inventor has discovered that the dielectric properties of tissue affected by an epidermal dysplastic lesion vary with respect to normal, healthy, tissue (i.e. tissue not affected by an epidermal dysplastic lesion). As such, the means for delivering microwave energy may electrically match the range of epsilon relative values of the tissue affected by an epidermal dysplastic lesion. In this way, it is possible to ensure efficient delivery of the microwave energy to the tissue.

The applicator (for delivering microwave energy) may not be inserted or implanted into the tissue of the subject to be treated.

The means for delivering the microwave energy to a subject may be connected to the microwave source via a flexible cable. In one embodiment the means for delivering the microwave energy to a subject (i.e. the applicator) may be connected to the microwave source via a flexible cable with locking connections having both microwave and signal data cables and may be reversible to enable connection to either port.

In one embodiment the invention provides an apparatus for delivering microwave energy to an epidermal dysplastic lesion the apparatus comprising:—a microwave source for providing microwave energy, connectable to a system controller for controlling at least one property of the microwave radiation provided by the microwave source; and a monitoring system for monitoring the delivery of energy and an applicator means, for example an applicator device, for delivering microwave energy, wherein:—the applicator is configured to deliver precise amounts of microwave energy provided by the source at a single frequency or across a range of frequencies.

A further embodiment of this invention provides a method of treating, curtailing or preventing an epidermal dysplasia condition (including any AK type lesion) including those conditions in which a tissue (for example the skin) is manifest with one or more dysplastic lesion(s), said method comprising administering a therapeutically effective and/or immunostimulatory dose or amount of microwave energy to the diseased tissue to produce, induce or elevate and immune response and/or levels of heat shock factor HSF to stimulate production of heat shock proteins in or near the tissue/dysplastic lesion. In particular, the method for treatment may produce, induce or elevate levels of one or more heat shock proteins selected from the group consisting of HSP90, HSP72, HSP70, HSP65. HSP60, HSP27, HSP16 and any another heat shock protein(s) wherein:—the microwave energy promotes an association between the elevated heat shock proteins and the infected tissue so as to elicit an immune response against the infection. Additionally or alternatively, the microwave energy may be sufficient to induce a local immune response which comprises modulated cell and cytokine induction/expression/activation and enhanced antigen presentation.

The energy deposited into the target tissues are primarily expressed as heat. The therapeutic temperature range for hyperthermia (heat shock initiation) is lower than that of ablation where cell death is the desired outcome. The embodiments of both modes, hyperthermia and ablation are covered in this invention. The choice of energy and subsequent mode of biological response can be selected by the clinician.

In a further embodiment of the invention there is provided a method of treating, curtailing or preventing an epidermal dysplastic lesion (including any AK type lesion) including those conditions in which a tissue (for example skin) is manifest with one or more dysplastic lesion(s), said method comprising administering a therapeutically effective and/or immunostimulatory dose or amount of microwave energy to diseased tissue to facilitate resolution or clearance of the epidermal dysplastic lesion and/or to cauterise, coagulate, shrink, block, ablate, damage, irritate, inflame or otherwise interfere with the normal operation of the capillaries supplying blood to the lesion. In one embodiment the lesion is a skin lesion including, for example, Actinic Keratosis.

In another embodiment, the present invention provides a medical treatment regime comprising:—the application of microwave energy to skin tissue, the skin tissue exhibiting symptoms associated with an epidermal dysplastic lesion, to purposefully elevate levels of heat shock factor HSF to stimulate production of heat shock proteins in or near the tissue in particular HSP90, HSP72, HSP70, HSP65. HSP60, HSP27, HSP16 and any another heat shock protein(s) wherein the microwave energy promotes an association between the elevated heat shock proteins and the diseased tissue with the intention of provoking an immune response against the dysplastic region.

### BRIEF DESCRIPTION OF THE DRAWINGS

Embodiments of the invention are now described, by way of non-limiting example, and are illustrated in the following figures, in which:—

FIG. 1 is a schematic illustration of an embodiment of a microwave treatment system.

FIG. 2(a) is a schematic illustration of a Papilloma caused by HPV infection.

FIG. 2(b) is a schematic illustration of the terminal differentiation pathway of epidermal cells infected by the HPV virus.

FIG. 3 is a schematic illustration of functional representation of a microwave treatment system for application to treat Papilloma or other dermatological lesions.

FIG. 4 is a schematic illustration of a microwave treatment system applicator according to an alternative embodiment.

FIG. 5 is a schematic illustration of the microwave activation of the heat shock response.

FIG. 6(a) is a schematic illustration of measurement results of Er vs. Frequency for verrucae tissue for a sample population.

FIG. 6(b) is a schematic illustration of results of Loss tangent vs. Frequency for verrucae tissue for a sample population.

FIG. 7 is a schematic illustration of Er vs. Frequency for various plantar tissues for a sample population.

FIG. 8 is a schematic illustration of the statistical analysis of the sample median of verrucae tissue for a sample population.

FIG. 9 is a schematic illustration of a comparison of the statistical analysis of sample medians of various plantar tissues for a sample population.

FIG. 10(A) is a Clinical image of plantar wart pre-microwave treatment (left), after one treatment (middle) and after two treatments (right). FIG. 10(B) is a clinical image of plantar wart pre-microwave treatment (left), after one treatment (right). FIG. 10(C) is a graphical representation of data from an intention to treat analysis, in which 32 patients with 54 HPV foot warts were treated by microwave therapy over 5 visits: baseline, 1 week, 1 month, 3 months, and 12 months. Resolved warts were enumerated.

FIG. 11(A) shows histological analysis of human skin treated with microwave visualised at the epidermis/papillary dermis (top and bottom panels), or deep dermis (middle panels). Skin was subject to microwave therapy (0-200 J), before punch excision. Tissue was cultured for 1 hour before fixation and paraffin embedding. H&E or TUNEL staining. Original magnifications, ×20. FIG. 11(B) shows histological analysis of human skin treated with microwave visualised at the epidermis/papillary dermis (upper panels), or deep dermis (lower panels). Skin was subject to various liquid nitrogen therapy for 5, 10, or 30 seconds, before punch excision. Tissue was cultured for 1 hour before fixation and paraffin embedding. H&E or TUNEL staining. Original magnifications, ×20. FIG. 11(C) is a graphical representation of data obtained following microwave therapy (top panel) or cryotherapy (bottom panel), skin samples (in triplicate) were excised and cultured in media for 1 or 16 hours before harvesting supernatant for measurement of lactate dehydrogenase (LDH) by ELISA. FIG. 11(D) shows images in which skin was subject to microwave therapy (150 J), before punch excision at the margin of the treated zone. Tissue was cultured for 1 hour before fixation and paraffin embedding. H&E stain. Original magnifications, ×10. FIG. 11(E) shows images in which skin was subject to microwave therapy (150 J), before punch excision. Tissue was cultured for 1 hour before fixation and paraffin embedding. H&E stain showing deep dermis. Original magnifications, ×100. Representative of 3 independent experiments.

FIG. 12(A), Left panel shows flow cytometric analysis of viable keratinocytes (% of total cells) indicated by negative staining with the amine reactive viability dye LIVE/DEAD after control, microwave (5-150 J), or LPS/IFNg treatment. Keratinocytes were treated, then kept in culture for 24, 48 or 72 hours before analysis. FIG. 12(A), Right panel shows flow cytometric analysis of keratinocyte viability after microwave therapy or control depicted as a histogram. X-axis: MFI LIVE/DEAD stain; y-axis: cell count. FIG. 12(B) shows flow cytometric analysis of HLA-DR, ICAM-1, CD40 or CD80 expression on viable keratinocytes. Keratinocytes were treated with microwave therapy (5-150 J), LPS/IFNg, or nil (control), rested in culture for 24, 48 or 72 hours, before analysis of the viable population. FIG. 12(C) shows flow cytometric analysis of CD86, CD80, and CD40 expression on viable monocyte derived dendritic cells (moDCs). Keratinocytes were treated with microwave therapy (5-150 J), LPS/IFNg, or nil (control), rested in culture for 8 hours then washed. They were left in culture for the remaining time until 24 or 48 hours, before transfer of supernatant onto moDCs. MoDCs were incubated for 24 hours before harvesting for analysis. Data representative of 3 independent experiments. Mean+SD.

FIG. 13(A) shows ELISpot assay of IFN-γ production by HPV-specific CD8+ cells following co-culture with HPV peptide pulsed moDCs primed by supernatant from untreated, microwave treated (150 J) or cryotherapy (cryo) treated keratinocytes. moDCs were primed with supernatant for 24 hours prior to pulsing with control or HPV peptide for 2 hours. Pulsed moDCs were then cocultured with an HLA-matched CD8+ HPV-specific T cell line before assay with IFN-γ ELISpot. Statistical significance determined using the Holm-Sidak method, with alpha=5%. Data representative of 3 independent experiments. Mean+SD. FIG. 13(B) shows ELISpot assay of IFN-γ production by HPV-specific CD8+ cells following co-culture with HPV E16 protein pulsed moDCs primed by supernatant from untreated, microwave treated (150 J) or cryotherapy (cryo) treated keratinocytes. moDCs were primed with supernatant for 24 hours prior to pulsing with control or HPV peptide for 2 hours. Pulsed moDCs were then co-cultured with an HLA-matched CD8+ HPV-specific T cell line before assay with IFN-γ ELISpot. Statistical significance determined using the Holm-Sidak method, with alpha=5%. Data representative of 3 independent experiments. Mean+SD. FIG. 13(C) shows flow cytometric analysis of intracellular HSP-70 expression on viable keratinocytes after microwave therapy or control depicted as a histogram. Primary human keratinocytes were treated with microwave therapy (150 J), or nil (untreated), rested in culture for 24 hours, before analysis. X-axis: MFI anti-HSP-70; y-axis: cell count. FIG. 13(D) shows ELISA of IL-6, TNFα, IL-1β production by primary human keratinocytes 24 h after treatment with microwave therapy (150 J), LPS/IFNg, cryotherapy or nil (untreated). FIG. 13(E) shows expression changes of IRF1 and IRF4 in human skin with microwave therapy (25 J and 150 J) by qPCR.

### DETAILED DESCRIPTION OF EMBODIMENTS

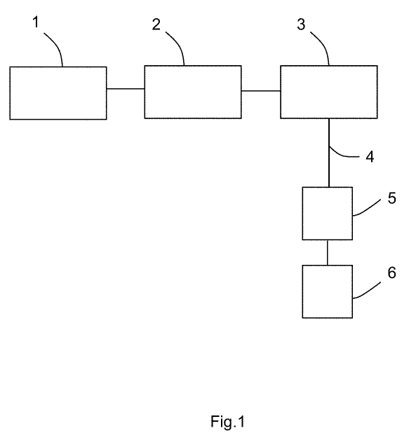

An embodiment of a microwave power generator system for medical applications is illustrated in FIG. 1 The apparatus comprising:—a microwave source for providing microwave energy 1, connectable to a system controller 2 for controlling at least one property of the microwave radiation provided by the microwave source; and a monitoring system 3 for monitoring the delivery of energy and an interconnecting cable 4 and an applicator hand piece 5 and a removable applicator means 6, for example an applicator device, for delivering microwave energy, wherein:—the applicator is configured to deliver precise amounts of microwave energy provided by the source at a single frequency or across a range of frequencies.

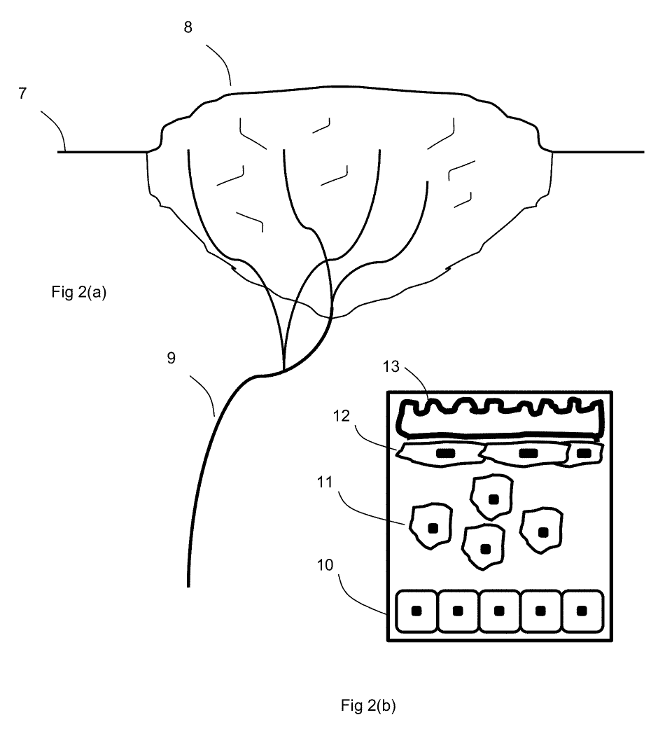

A typical HPV infection is illustrated in FIG. 2(a), this comprises the normal tissue 7, the papilloma surface 8, the capillary feed network 9. The terminal differentiation pathway of epidermal cells infected by the HPV virus is illustrated in FIG. 2(b) basal cells 10 become infected with the HPV virus leading to viral replication in the stratum spinosum 11 followed by assembly of virus particles in the stratum granulosum and release of virus particles in the in the stratum corneum (papilloma surface).

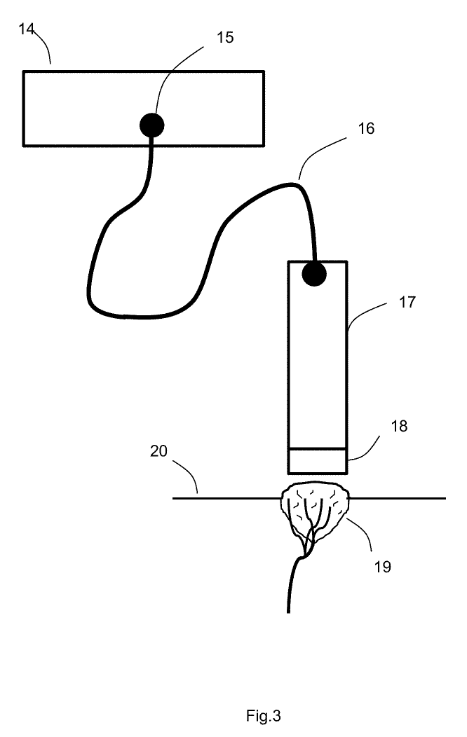

FIG. 3 shows the components of an apparatus according to an embodiment of the present invention, the components shown separately for ease of reference. The apparatus comprises a generator system 14 with a locking microwave connection 15 to a flexible microwave cable 16 connected to a hand piece 17 (which may have the same type of locking connection) which accepts an applicator component 19. The applicator component is designed to match to the tissue properties of the papilloma 20 and not match to the normal tissue 18. The cable 16 may include both microwave and signal data cables and may be reversible to enable connection to either port.

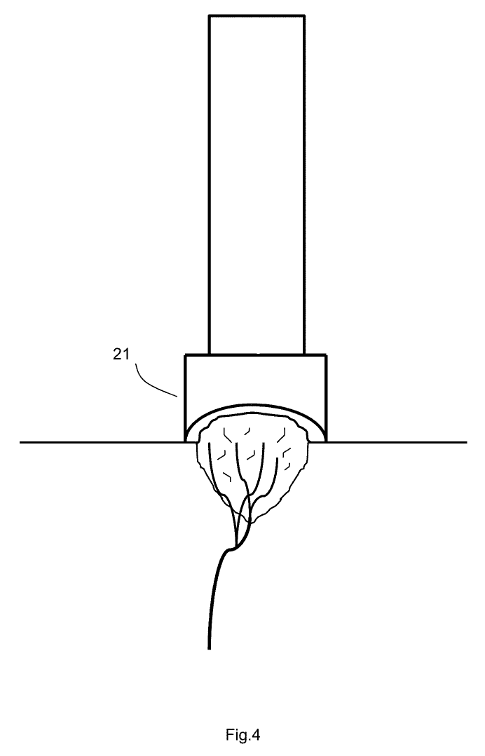

An alternative embodiment of an applicator 21 is illustrated in FIG. 4 with the component having a domed or enclosing surface compatible with raised or curved lesions.

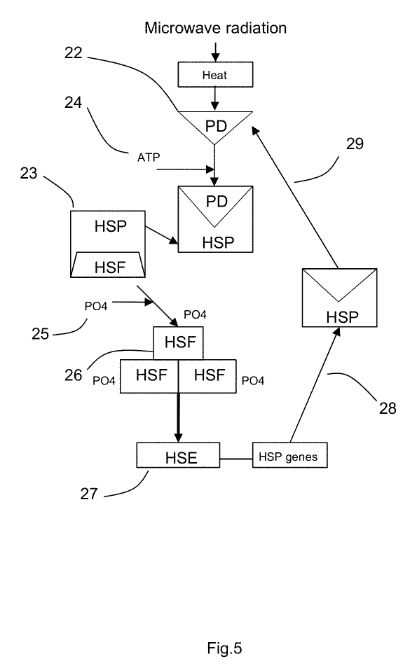

The method of inducing a microwave heat shock responses is illustrated in FIG. 5. In this illustration microwave radiation creates a thermal stress which results in protein denaturation 22. Heat shock proteins (HSP) are normally bound to heat shock factors (HSF) (23), but dissociate in the presence of denatured proteins (PD). Once dissociated, HSPs bind to the denatured proteins by rapid release. This requires Adenosine Triphosphate (ATP) 24. Further HSPs are generated when HSFs phosphorylate (PO4) (25) and trimerize (26). These trimers bind to heat shock elements (HSE) 27 that are contained within the promoters of the HSPs and generate more protein 28. Newly generated HSPs can then free to bind more denatured proteins 29.

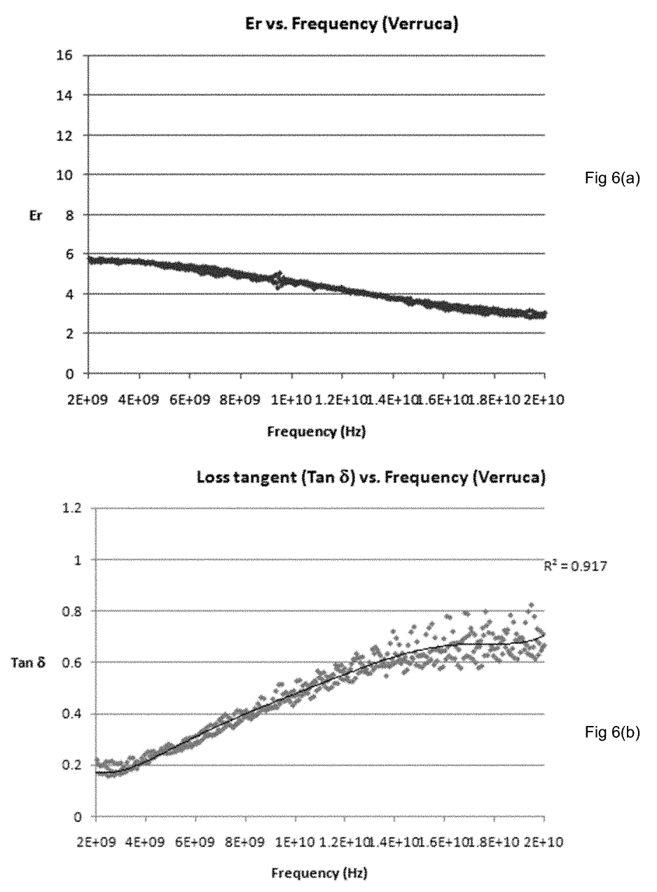

The measured dielectric properties for the sample population median versus frequency of plantar verrucae are reported in FIGS. 6(a) and 6(b). Measured results for ER and loss tangent versus frequency from 2 GHz to 20 GHz are reported. The measurements were made using an Agilent PNA-L Network Analyzer connected to an Agilent 85070E dielectric measurement system with the 85070E Performance Probe Kit (Option 050) measuring from 300 kHz to 20 GHz. Deionised water and air were used as dielectric references for calibration.

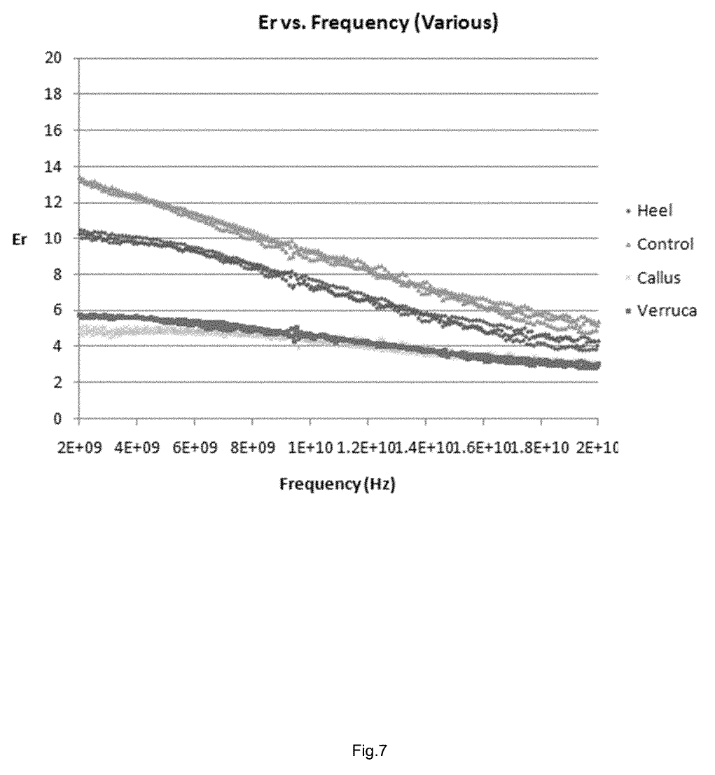

The measured dielectric properties for the sample population (median taken across the population) versus frequency for various plantar tissues are reported in FIG. 7. Measured results for Er versus frequency from 2 GHz to 20 GHz are reported illustrating demarcation between each tissue type.

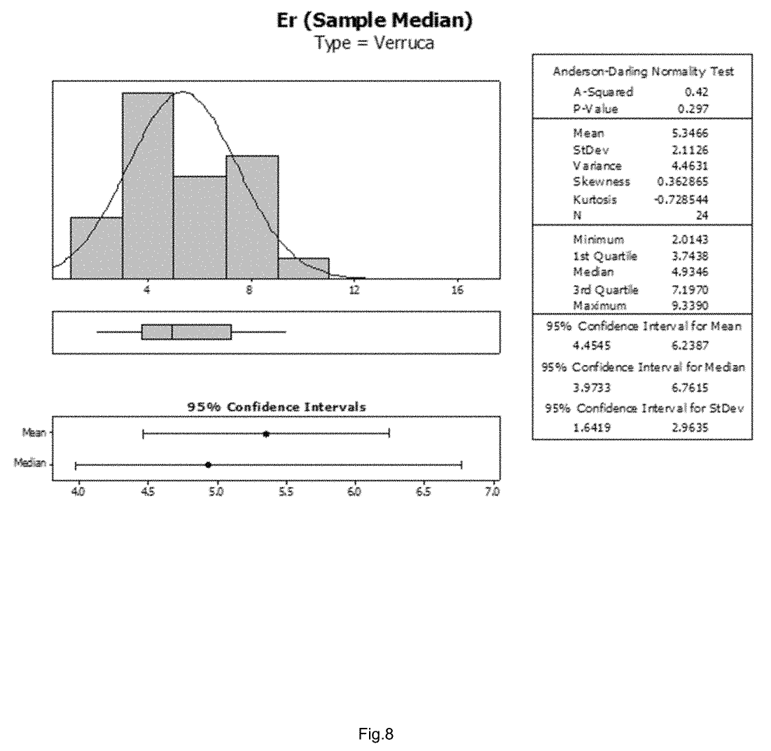

Statistically analysed dielectric property values taken over a sample population (using the median of the measurement range 7.5-8.5 GHz taken from each sample) for Verrucae tissue is presented in FIG. 8. The median Er value was measured at 4.93 for this dataset.

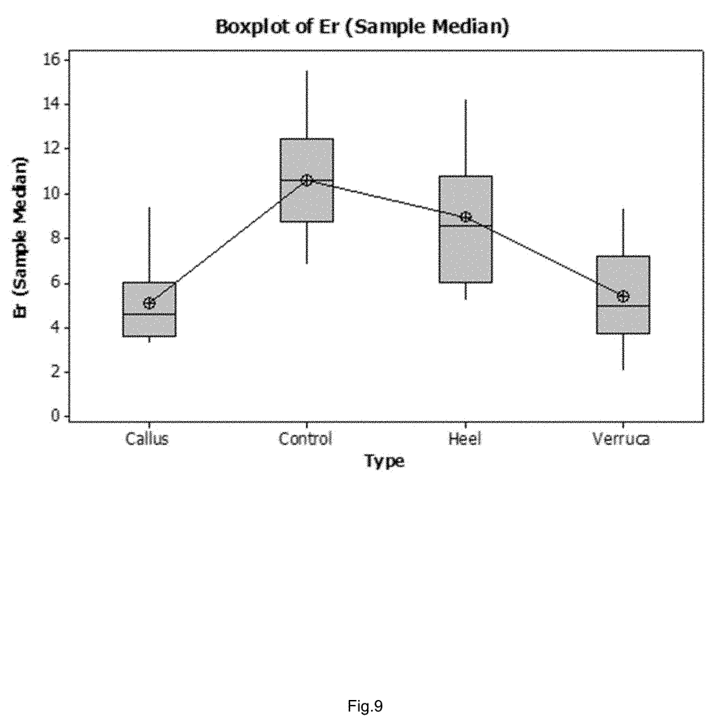

With reference to FIG. 9 a comparison of statistically analysed measurements of the dielectric properties of various plantar tissues over a sample population (using the median of the measurement range 7.5-8.5 GHz taken from each sample) is illustrated.

### Microwave Therapy for Cutaneous Human Papilloma Virus Infection

### Methods and Materials

### Patients and In Vivo Microwave Treatment

Patients with treatment-refractory plantar warts were excluded if they had a pacemaker fitted, were pregnant or breast feeding, had any metal implants within the foot or ankle, suffered any known disease or condition affecting their immune function or their capacity to heal. Adverse events were categorised as being specifically associated with the microwave procedure, or unrelated. A complete examination of the affected area was undertaken at each study visit. At the conclusion of the treatment session all patients were given an advice information sheet advised to report any complications. No post-operative dressing was required and patients were advised to subsequently undertake normal everyday activities as usual with no restrictions.

A total of 32 patients with 54 foot warts were enrolled into the study. Of the 32, 17 were males and 15 females. Ages ranged from 22-71 years with a mean age of 44.79 years (sd 13.019]. Of the 54 lesions, 16 were reported as single lesions, and 38 as multiple type lesions (including mosaic verrucae). The average lesion duration was 63 months (5.25 years) with a range of 2-252 months (<1-21 years). The mean lesion diameter was 7.43 mm (sd 6.021), ranging from just 2 mm to 38 mm in diameter.

The procedure was performed in an out-patient setting, with standard podiatric facilities. The Swift device settings were titrated up as tolerated to 50 J over a 7 mm² application area (7.14 j/mm²). The microwave energy was delivered to the affected area over 5 s duration (50 J delivered as 10 watts for 5 s). Lesions which were <7 mm in diameter were treated with one application of the probe at a single treatment session whilst lesions >7 mm were underwent multiple applications until the entire surface of the wart had been treated.

Clinical assessments were performed at baseline and at 1 week, 1 month, 3 months, and 12 months after treatment by a podiatrist experienced in the management of plantar warts. Response to treatment was assessed by the same investigator as ‘completely resolved’ or ‘unresolved’. Complete resolution was indicated by fulfilling three criteria: i. lesion no longer visible, ii. return of dermatoglyphics to the affected area, iii. no pain on lateral compression. Pain was assessed using a 10 point visual analogue scale.

### Human Skin and Ex Vivo Microwave Treatment

Normal skin samples were acquired from healthy individuals after obtaining informed written consent with approval by the Southampton and South West Hampshire Research Ethics Committee in adherence to Helsinki Guidelines. Skin samples were treated immediately ex-vivo with microwave (Swift s800; Emblation Ltd., UK) or liquid nitrogen therapy and treated skin excised. Excised skin was sent for histological analysis or placed in culture media.

Histological analysis with hematoxylin and eosin (H&E) tissue sections were undertaken following fixation and embedded in paraffin wax. DNA damage was assessed by staining for single stranded and double stranded DNA breaks by TUNEL assay using the ApopTag® In Situ Apoptosis Detection Kit (Millipore, UK). Following culture, supernatants were collected and analysed for lactate dehydrogenase release using the Cytotoxicity Detection Kit (Roche applied science) as a measure of apoptosis.

### Culture and In Vitro Microwave Treatment.

Human skin and HaCaT keratinocytes were cultured in calcium-free DMEM (ThermoFisher Scientific) with 100 U/mL penicillin, 100 μg/mL streptomycin, 1 mM sodium pyruvate, 10% fetal bovine serum (FBS) and supplemented with calcium chloride at 70 μM final concentration.

Lymphocytes were cultured in RPMI-1640 media with 100 U/mL penicillin, 100 μg/mL streptomycin, 1 mM sodium pyruvate, 292 μg/mL L-glutamine, supplemented with 10% FBS or 10% heat inactivated human serum (HS). HaCaT cells were cultured at sub-confluency to avoid cell differentiation and used in assays at passage 60-70. Cells were plated at 2.5×103 cells/well in 96-well flat plate (Corning Costar) and cultured overnight to reach confluence. HaCaTs were washed once with PBS before treatment with 150 J microwave, liquid nitrogen (10 s), heat (42° C. preheated media) or with LPS+IFN-γ (1 ng/mL+1000 U/mL). Cells were cultured for 24 h before supernatants were harvested.

For HPV-specific T cell lines, PBMCs were isolated from HLA-A2 individuals as previously described 11. PBMCs were seeded at 2-4×106 cells/well in 24-well culture plate and 10 μg/mL of 9mer HLA-A2 restricted HPV16 epitope LLM (LLMGTLGIV) 12 was added, cells were cultured in 1 mL RPMI+10% HS. On day 3, cells were fed with RPMI+10% HS+IL-2 (200 IU/mL), and then fed again on day 7 or when needed. After day 10, HPV-specific T cells were harvested for cryopreservation before testing against HPV in ELISpot assays.

Monocyte derived dendritic cells (moDCs), CD14+ cells were positively isolated from PBMCs by magnetic separation using CD14 microbeads (Milentyi Biotec), according to manufacturer's protocol. Cells were washed and resuspended in RPMI+10% FBS+250 U/mL IL-4 and 500 U/mL GM-CSF. At day 3, cells were fed with RPMI+10% FBS+IL-4 and GM-CSF, and then harvested on day 5 for use in functional assays.

In vitro, microwave therapy of cell cultures was delivered through the base of the plastic culture dish and showed a linear dose response between the energy delivered and thermal induction (not shown). Utilising the equation E=m×c×θ (E=energy transferred, J; m=mass, kg; c=specific heat capacity, J/kg ° C.; θ=temperature change, ° C.), we calculated that in our system the 150 J Swift programme delivered 15.58 J (s.d. 0.921) through the plastic to the culture.

ELISpot, Flow Cytometry and qPCR

Keratinocytes were treated with microwave at various energy settings before removal of supernatant at various time points. MoDCs were treated overnight with keratinocyte supernatant, then washed twice before incubation with 10 μg/mL LLM peptide for 2 hours before a further wash.

Human IFN-γ ELISpot (Mabtech, Sweden) was undertaken as per manufacturer's protocol and as reported previously 11. 1×103 moDCs were plated with autologous HPV peptide-specific T cells at 1:25 ratio. Spot forming units (sfu) were enumerated with ELISpot 3.5 reader (AID, Germany). MoDCs were treated with HaCaT supernatant and harvested at 24 hours for flow cytometric analysis of cell phenotype. Cells were stained with violet LIVE/DEAD stain (Invitrogen) for 30 min at 4° C., then washed with PBS+1% BSA and stained with antibodies PerCP-Cy5.5 anti-HLA-DR, FITC anti-CD80, FITC anti-CD86, PE anti-CD40, all purchased from BD, for 45 min at 4° C. Cells were washed then resuspended in PBS+1% BSA and analysed using the BD FACSAria and the FlowJo v10.0.08 analysis software.

The expression of chosen genes was validated with quantitative PCR, using the TaqMan gene expression assays for target genes: YWHAZ (HS03044281_g1), IRF1 (Hs00971960_m1), IRF4 (Hs00543439_CE) (Applied Biosystems, Life Technologies, Paisley, UK) in human skin treated as indicated. RNA extraction (RNeasy micro kit, Qiagen) and reverse transcription (NanoScript kit; Primer Design, Southampton, UK) were carried out accordingly to the manufacturer's protocol.

### Results:

Treatment of Human Papilloma Virus Infection in Humans with Microwave Therapy

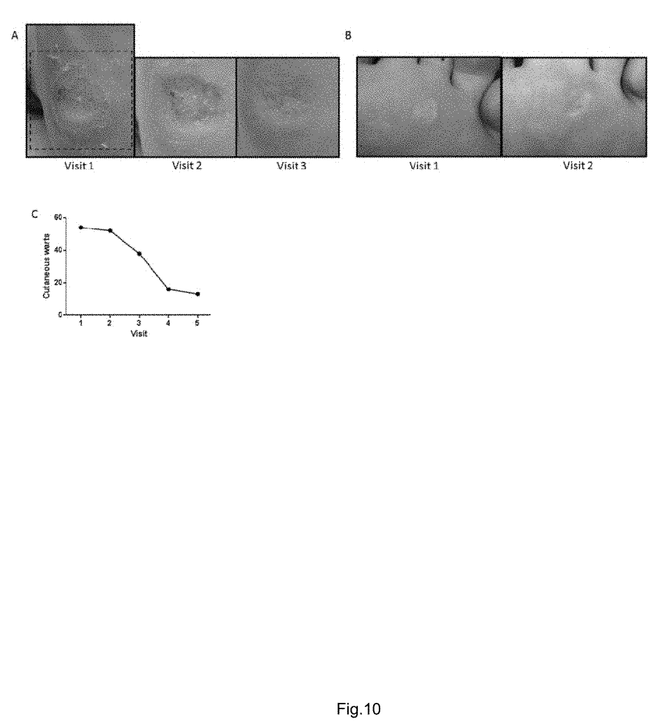

From January 2015 to September 2015 at the University of Southampton, we enrolled 32 patients with severe, treatment-refractory plantar warts. The diagnosis of plantar wart was confirmed by a podiatrist experienced in management of such lesions. A clinically significant wart was defined as >1 year duration, which had failed at least two previous treatments (salicylic acid, laser, cryotherapy, needling and surgical excision). In each patient, the most prominent plantar wart (most severely affected) was targeted for treatment (FIG. 10 a,b).

At the end of the study period, of the 54 warts treated, 41 had resolved (75.9%), 9 remained unresolved (16.7%), 3 warts (n=2 patients) had withdrawn from the study (5.6%) and 1 patient (with 1 wart) was lost to follow up (1.9%). The mean number of days to resolution 79.49 days (sd 34.561; 15-151 days). 94% of resolving lesions had cleared after 3 treatments (FIG. 10c). No significant difference in resolution rates were observed between males and females (p=0.693) was observed.

### Microwave Treatment of Human Skin

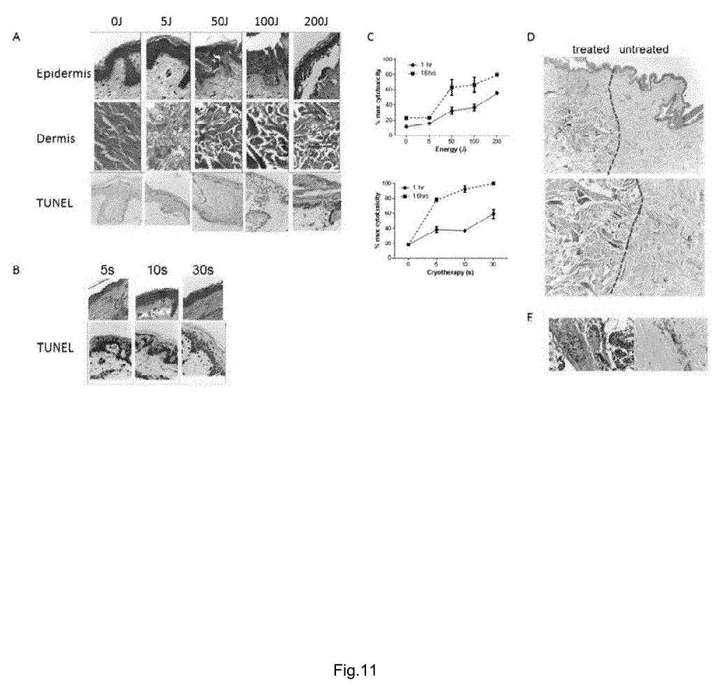

Human skin has not been previously treated with microwave therapy, therefore, we proceeded to undertake a full histological analysis. Skin removed during routine surgery was sectioned 1 hour after treatment ex vivo. Neither macroscopic, nor histological changes were noted with the lowest energy setting (5 J). At 50 J, mild macroscopic epidermal changes only were noted, and microscopically minor architectural changes, and slight elongation of keratinocytes were seen without evidence of dermal collagen sclerosis. At higher energies (100, 200 J) gross tissue contraction was visible macroscopically. Microscopic changes in the epidermis were prominent, showing spindled keratinocytes with linear nuclear architectural changes and subepidermal clefting (FIG. 11a).

Dermal changes were prominent at energies of 100 J and above and showed a homogenous zone of papillary dermal collagen, thickened collagenous substances, accentuation of basophilic tinctorial staining of the dermal collagen with necrotic features (FIG. 11a). These features are similar to electrocautery artefacts and suggest the potential to induce scarring at >100 J. Histological analysis both at 16 h and 45 h showed similar changes (not shown).

In clinical practice, cryotherapy is delivered to the skin by cryospray, which is time-regulated by the operator. In contrast to microwave therapy, minimal epidermal or dermal architectural change was identified with cryotherapy at standard treatment duration times (5-30 s), but did show a dose dependent clumping of red blood cells in vessels (FIG. 11b).

Tissue release of LDH acts as a biomarker for cellular cytotoxicity and cytolysis. To examine the extent of cell death induced by microwave irradiation, human skin was treated with 0, 50, 100 or 200 J before punch excision of the treated area and incubation in medium for 1 hour or 16 hours. Measurement of LDH revealed a dose dependent induction of tissue cytotoxicity with increasing microwave energies (FIG. 11c). In line with the lack of histological evidence of cellular damage, at 5 J, cytotoxicity of microwave application was equivalent to control. Early cytotoxicity was not prominent at 50 J, but became more evident after 16 hours. Higher energy levels induced more prominent cytotoxic damage. In contrast to microwave therapy, liquid nitrogen treatment of skin induced cytotoxicity at the lowest dose both at 1 hour and 16 hours.

Terminal deoxynucleotidyl transferase dUTP nick end labelling (TUNEL) identifies cells in the late stage of apoptosis. Analysis at 0, 5, 50, 100 and 200 J identified increased cellular apoptosis in the epidermis above 100 J (FIG. 11a). In contrast, cryotherapy with a liquid nitrogen spray applicator directly to the skin as used in clinical practice, even at a very short treatment time (5 s) induced significant epidermal and dermal DNA fragmentation (FIG. 11b).

The physics of microwave therapy suggests a tight boundary between treated and untreated tissue with minimal spreading of the treated field. This was borne out histologically by a clear demarcation between treated areas extending vertically from the epidermis through the dermis (FIG. 11d). Examination of the dermis showed that microwave therapy modified skin adnexae inducing linear nuclear architectural changes in glandular apparatus, microthrombi, fragmented fibroblasts and endothelial cells (FIG. 11e).

### Microwave Induction of Immune Responses in Skin

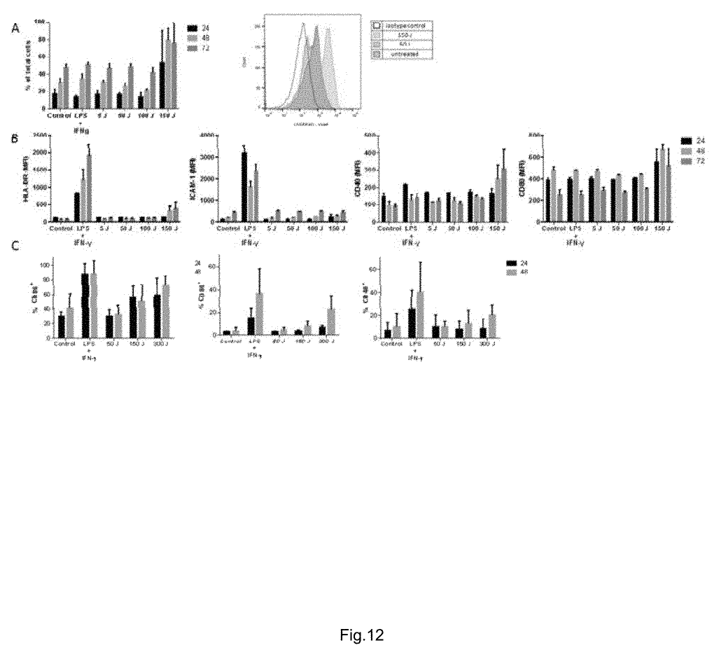

We first examined the response of keratinocytes to microwave therapy in vitro. Keratinocyte apoptosis was induced by microwave therapies above 100 J in vitro (FIG. 12a). Only above the apoptotic threshold were surface phenotypic changes of cellular activation noted in viable cells with increased expression of HLA-DR, CD40 and CD80 (FIG. 12b). Next, we utilised a model of skin cross talk of keratinocyte signalling to dermal dendritic cells. Keratinocytes were treated with microwave therapy as above, and washed after 8 hours to remove dead or apoptotic cells. Treated keratinocytes were then incubated for a further 16 hours before supernatant collection to prime monocyte derived dendritic cells (moDCs) which had not been directly exposed to microwave therapy. This showed a potent induction of moDC activation with increased expression of CD86, CD80 and to a lesser extent CD40 (FIG. 12c).

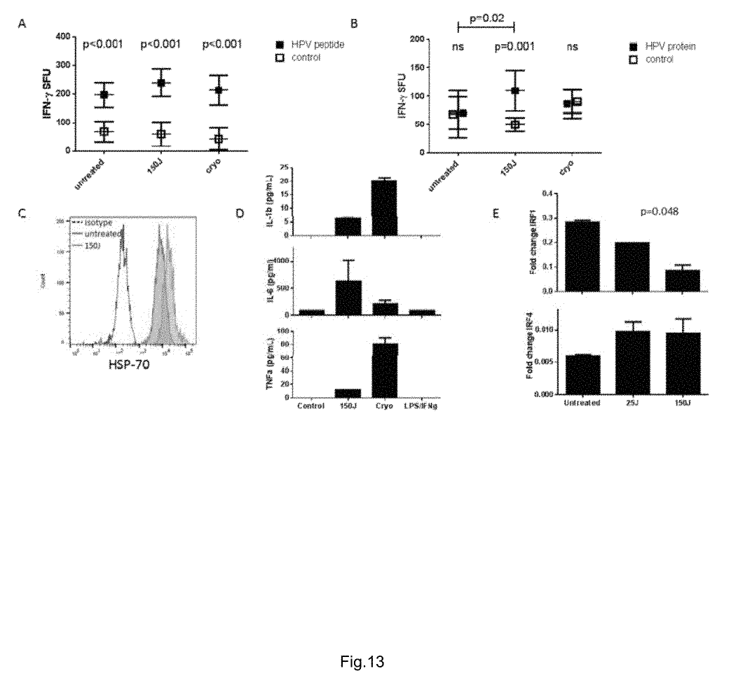

We next set out to test the functional outcome on skin dendritic cells following microwave treatment of keratinocytes. Keratinocytes were untreated, microwave, or cryotherapy treated before supernatant harvesting. Supernatant primed DCs were pulsed with a 9 amino acid HLA-A2 epitope (LLM) from human papilloma virus (HPV) E16 protein and cultured with an autologous HPV specific CD8+ T cell line. As expected, in all conditions, the DCs efficiently presented HPV peptide to CD8+ T cells inducing IFN-γγ (FIG. 13a). However, dendritic cell presentation of HPV virus is dependent upon cross-presentation to the MHC class I pathway. Therefore we also tested the ability of untreated, microwave treated or cryotherapy KC-primed DCs to present human papilloma virus (HPV) E16 protein to an HLA matched HPV-specific CD8+ T cell line. Strikingly, only microwave treated KCs were able to prime DCs to enhance cross-presentation (FIG. 13b). To explore the potential mechanism of keratinocyte response to microwave therapy we confirmed up regulation of HSP-70 in response to microwave therapy of keratinocytes (FIG. 13c) and showed significant IL-6 induction in keratinocytes above that seen in cryotherapy treated cells (FIG. 13d). IRF1 and IRF4 are key regulators of dendritic cell activation and we confirmed that microwave therapy induced down regulation of IRF1 and up-regulation of IRF4 (FIG. 13e).

### Discussion

This is the first study to investigate the potential efficacy of locally delivered microwaves in the treatment of cutaneous viral warts in vivo. We report a complete resolution rate of 75.9% recalcitrant plantar warts (average lesion duration of over 5 years). This compares very well with previous reports of plantar wart resolution for salicylic acid and or cryotherapy (23-33%)¹³. Whilst this study was a pilot phase, and did not include a control untreated arm, we believe the treatment effect to be significant.

For all novel therapies, adverse events are critical. In this study we did not identify a strong signal for adverse events with microwave therapy of cutaneous warts. As with current physical treatments for warts discomfort is expected for the patient. During the study patients typically reported that for a typical 5 second treatment that they endured moderate discomfort for approximately 2 seconds, which immediately diminished after the treatment had completed. In addition, it was commonly noted that discomfort was less with subsequent treatments. One male patient, withdrew from the study after one treatment, citing the pain of treatment as the reason. In the study design phase, preoperative use of topical anaesthetic cream was tested, but appeared to do little to mitigate the pain (unpublished data) and it was felt that the pain of local anaesthetic injection would exceed that normally experienced during a microwave treatment. Following microwave therapy, patients did not require dressings or special advice as microwave therapy utilised in this study did not cause a wound or ulcer in the skin, allowing the patient continue normal activity. The short microwave treatment time (5 s) offers a significant clinical advantage over current wart therapies such as cryotherapy and electro-surgery. Within 5 s, microwaves penetrate to a depth of over 3.5 mm at the energy levels adopted for the study¹⁴—possibly a greater depth than can be attained by cryosurgery or laser energy devices. Moreover, as microwaves travel in straight lines energy is deposited in alignment the device tip with little collateral spread, meaning minimal damage to surrounding tissue, as observed in this study. Microwaves induce dielectric heating. When water, as a polar molecule, is exposed to microwave energy, the molecule is excited and rotates attempting to align with the alternating electro-magnetic field. At microwave frequencies the molecule is unable to align fully with the continuously shifting field resulting in heat generation. Within tissues, this acts to rapidly elevate temperatures. This process rapidly changes cellular heat because it does not depend on tissue conduction. Microwave treatment produces no vapour or smoke unlike ablative lasers and electro-surgery, eliminating the need for air extraction systems due to the risk of spreading viral particles within the plume¹⁵.

Although, microwave therapy has been considered a tissue ablation tool, we saw minimal skin damage after treatment with 50 J, yet apparent good clinical response. Therefore we investigated whether there was evidence to support an induction of immunity by microwave therapy. The critical nature of CD8+ T cell immunity for host defence against HPV skin infection is well established and supported by the observation of increased prevalence of infection in immunosuppressed organ transplant recipients¹⁶, and that induction of protection from HPV vaccines is mediated by CD8+ T cells¹⁷. We show here, that microwave therapy of skin induces keratinocyte activation and cell death through apoptosis. However, at sub-apoptotic doses, microwave primed keratinocytes are able to signal to dendritic cells and enhance cross-presentation of HPV antigens to CD8+ lymphocytes which offers a potential explanation for the observed response rate in our clinical study. In vitro evidence suggests that this is likely to be mediated by cross-talk between microwave treated skin keratinocytes and dendritic cells, with resultant enhanced cross-presentation of HPV protein to CD8+ T cells. Microwave therapy also induced enhanced IL-6 synthesis from keratinocytes.

IL-6, is a pro-inflammatory mediator, important in anti-viral immunity which has been recently shown to induce rapid effector function in CD8+ cells¹⁸. Thus, IL-6 up-regulation may provide an important additional mechanism for microwave anti-viral immunity. IRFs have been shown to be central to the regulation of immune responses¹⁹⁻²¹. IRF4 is essential for differentiation of cytotoxic CD8+ T cells²²²³, but up-regulation in dendritic cells has also been shown to enhance CD4+ differentiation, thereby potentially enhancing both CD8+ immunity and T cell help following microwave treatment. IRF1 expression has been previously reported to be modulated by HPV infection, but different models have shown opposite outcomes^{24,25}. We show down-regulation of IRF1 in human skin in association with a microwave therapy which supports the proposal of IRF-1 as a therapeutic target in HPV infection²⁵.

This study is the first of its kind studying microwaves in the treatment of plantar warts in vivo. However, the authors acknowledge the limitations of the uncontrolled, non-randomised design. Despite the promising results shown here, studies with larger sample sizes are needed to assess the efficacy of this treatment and for infrequent but serious adverse events.

It will be understood that embodiments of the present invention have been described above purely by way of example, and modifications of detail can be made within the scope of the invention.

Each feature disclosed in the description, and (where appropriate) the claims and drawings may be provided independently or in any appropriate combination.

### REFERENCES

1. Cockayne S, Hewitt C, Hicks K, et al. Cryotherapy versus salicylic acid for the treatment of plantar warts (verrucae): a randomised controlled trial. BMJ 2011; 342:d3271.
2. Stern P L. Immune control of human papillomavirus (HPV) associated anogenital disease and potential for vaccination. Journal of clinical virology: the official publication of the Pan American Society for Clinical Virology 2005; 32 Suppl 1:S72-81.
3. Soong R S, Song L, Trieu J, et al. Toll-like receptor agonist imiquimod facilitates antigenspecific CD8+ T-cell accumulation in the genital tract leading to tumor control through IFNgamma. Clin Cancer Res 2014; 20:5456-67.
4. Edwards L, Ferenczy A, Eron L, et al. Self-administered topical 5% imiquimod cream for external anogenital warts. HPV Study Group. Human Papilloma Virus. Arch Dermatol 1998; 134:25-30.
5. Bristow I, Walker N. Pulsed Dye laser for the treatment of plantar warts—two case studies. Foot 1997; 7:229-30.
6. Kimura U, Takeuchi K, Kinoshita A, Takamori K, Suga Y. Long-pulsed 1064-nm neodymium:yttrium-aluminum-garnet laser treatment for refractory warts on hands and feet. The Journal of Dermatology 2014; 41:252-7.
7. Park H, Choi W. Pulsed dye laser treatment for viral warts: A study of 120 patients. Journal of Dermatology 2008; 35:491-8.
8. Tosti A, Piraccini B M. Warts of the Nail Unit: Surgical and Nonsurgical Approaches. Dermatol Surg 2001; 27:235-9.
9. Sterling J C, Gibbs S, Haque Hussain S S, Mohd Mustapa M F, Handfield-Jones S E. British Association of Dermatologists' guidelines for the management of cutaneous warts 2014. Br J Dermatol 2014; 171:696-712.
10. Lloyd D M, Lau K N, Welsh F, et al. International multicentre prospective study on microwave ablation of liver tumours: preliminary results. HPB: the official journal of the International Hepato Pancreato Biliary Association 2011; 13:579-85.
11. Polak M E, Thirdborough S M, Ung C Y, et al. Distinct molecular signature of human skin Langerhans cells denotes critical differences in cutaneous dendritic cell immune regulation. J Invest Dermatol 2014; 134:695-703.
12. Ressing M E, de Jong J H, Brandt R M, et al. Differential binding of viral peptides to HLA-A2 alleles. Implications for human papillomavirus type 16 E7 peptide-based vaccination against cervical carcinoma. European journal of immunology 1999; 29:1292-303.
13. Bruggink S C, Gussekloo J, Berger M Y, et al. Cryotherapy with liquid nitrogen versus topical salicylic acid application for cutaneous warts in primary care: randomized controlled trial. CMAJ 2010; 182:1624-30.
14. Emblation Medical Limited. Swift applicator instructions for use. Alloa, Scotland 2012.
15. Karsai S, Daschlein G. “Smoking guns”: Hazards generated by laser and electrocautery smoke. J Dtsch Dermatol Ges 2012; 10:633-6.
16. Tan H H, Goh C L. Viral infections affecting the skin in organ transplant recipients: epidemiology and current management strategies. Am J Clin Dermatol 2006; 7:13-29.
17. de Jong A, O'Neill T, Khan A Y, et al. Enhancement of human papillomavirus (HPV) type 16 E6 and E7-specific T-cell immunity in healthy volunteers through vaccination with TA-CIN, an HPV16 L2E7E6 fusion protein vaccine. Vaccine 2002; 20:3456-64.
18. Bottcher J P, Schanz O, Garbers C, et al. IL-6 trans-signaling-dependent rapid development of cytotoxic CD8+ T cell function. Cell reports 2014; 8:1318-27.
19. Schlitzer A, McGovern N, Teo P, et al. IRF4 transcription factor-dependent CD11b+ dendritic cells in human and mouse control mucosal IL-17 cytokine responses. Immunity 2013; 38:970-83.
20. Vander Lugt B, Khan A A, Hackney J A, et al. Transcriptional programming of dendritic cells for enhanced MHC class II antigen presentation. Nat Immunol 2013.
21. Tussiwand R, Lee W L, Murphy T L, et al. Compensatory dendritic cell development mediated by BATF-IRF interactions. Nature 2012; 490:502-7.
22. Huber M, Lohoff M. IRF4 at the crossroads of effector T-cell fate decision. European journal of immunology 2014; 44:1886-95.
23. Raczkowski F, Ritter J, Heesch K, et al. The transcription factor Interferon Regulatory Factor 4 is required for the generation of protective effector CD8+ T cells. Proc Natl Acad Sci USA 2013; 110:15019-24.
24. Park J S, Kim E J, Kwon H J, Hwang E S, Namkoong S E, Um S J. Inactivation of interferon regulatory factor-1 tumor suppressor protein by HPV E7 oncoprotein. Implication for the E7-mediated immune evasion mechanism in cervical carcinogenesis. J Biol Chem 2000; 275:6764-9.
25. Muto V, Stellacci E, Lamberti A G, et al. Human papillomavirus type 16 E5 protein induces expression of beta interferon through interferon regulatory factor 1 in human keratinocytes. Journal of virology 2011; 85:5070-80.

### ADDITIONAL REFERENCES

1. https://emedicine.medscape.com/article/1099775-treatment
2. https://www-clinical key-com.rsm.idm.ocic.org/#!/content/book/3-s2.0-B978070205183800031X?scrollTo=%23hl0001716
3. https://www.ncbi.nlmnih.gov/pmc/articles/PMC4065271/
4. http://www.bad.org.uk/for-the-public/patient-information-leaflets/actinic-keratoses/?showmore=1&returnlink=http%3A%2F%2Fwww.bad.org.uk%2Ffor-the-public%2Fpatient-information-leaflets#.Wn2AyiXFKHt
5. http://www.pcds.org.uk/clinical-guidance/actinic-keratosis-syn.-solar-keratosis

1. A method of treating at least one of:
(i) dysplastic epidermal lesions; and/or
(ii) a dermatological pre-cancerous disease;
said method comprising administering to a subject having said at least one of a dysplastic epidermal lesion and/or dermatological disease, a therapeutically effective and/or immunostimulatory amount or dose of microwave energy.

2. The method of claim 1, wherein the treatment comprises repeated rounds of treatment with microwave energy.

3. The method of claim 1, wherein the dysplastic epidermal lesion and/or dermatological disease is at least one of:
a. Actinic keratosis;
b. Solar keratosis;
c. Actinic cheilitis;
d. Arsenical keratosis;
e. PUVA keratosis;
f. Dysplastic lesion
g. Pre-cancerous dermatological disease

4. The method of claim 1, wherein the microwave energy has a frequency selected from the group consisting of:
(i) between about 500 MHz and about 200 GHz;
(ii) between about 900 MHz and about 100 GHz;
(iii) between about 5 GHz to about 15 GHz.

5. The method of claim 1, wherein the microwave energy has a frequency of about 8 GHz.

6. The method of claim 1 wherein the dose or amount of microwave energy produces, induces or elevates levels of heat shock factor (HSF) to stimulate production of a heat shock protein within a tissue or lesion being treated.

7. The method of claim 6, wherein the heat shock protein is selected from the group consisting of HSP90, HSP72, HSP70, HSP65. HSP60, HSP27, HSP16 and any another heat shock protein(s).

8. The method of claim 8, wherein the microwave energy promotes an association between the elevated heat shock protein and the dysplastic tissue so as to elicit an immune response against the disease.

9. The method of claim 1, wherein the subject is suffering from a viral lesion and the method is in part applied for the treatment or prevention of a viral lesion, said method comprising delivering a therapeutically effective amount or dose of microwave energy to the lesion, wherein the microwave energy causes the denaturing of viral particles within the lesion thus exposing antigenic sites stimulating an immune response.

10. The method of claim 9, wherein the viral lesion is a viral skin lesion comprising at least one of an actinic keratosis, solar keratosis or dysplastic lesion.

11. An apparatus for use in treating a dysplastic epidermal lesion, said apparatus comprising a microwave source for providing microwave energy and a delivery system for delivering the microwave energy to a subject to be treated.

12. The apparatus of claim 11, further comprising at least one of:
(i) a controller for controlling at least one property of the microwave energy produced by the microwave source; and/or
(ii) a monitor for monitoring the microwave energy produced by the microwave source.

13. The apparatus of claim 11, wherein the delivery system for delivering microwave energy electrically matches the range of epsilon relative values of the tissue affected by said at least one dysplastic epidermal lesion.

14. The apparatus of claim 11, wherein the delivery system for delivering the microwave energy to a subject comprises a component for contact with a subject to be treated.

15. The apparatus of claim 14, wherein the component is removable such that it can be discarded or sterilised after use.

16. A method of treating or preventing dysplastic epidermal lesions and/or a dermatological pre-cancerous disease, said method comprising administering to a subject having said dysplastic epidermal lesion and/or a dermatological pre-cancerous disease, or susceptible/predisposed to developing the same, a therapeutically effective amount or dose of microwave energy to the dysplastic epidermal lesion and/or dermatological pre-cancerous disease; or to a site vulnerable to the development of the same, wherein:
the administering of the therapeutically effective amount or dose of microwave energy comprises using a delivery system to administer the therapeutically effective amount or dose of microwave energy via a microwave applicator to tissue of the subject that has dysplastic epidermal lesions and/or a dermatological pre-cancerous disease or is susceptible/predisposed to the development of the same.

17. The method of claim 16, wherein the method comprises electrically matching the microwave applicator to the tissue that has said dysplastic epidermal lesion and/or a dermatological pre-cancerous disease, based on the tissue that has said dysplastic epidermal lesion and/or a dermatological pre-cancerous disease having a lower dielectric constant than a dielectric constant that said tissue would have if said tissue did not have said dysplastic epidermal lesion and/or a dermatological pre-cancerous disease, such that the microwave applicator is better matched to the tissue that has said dysplastic epidermal lesion and/or a dermatological pre-cancerous disease than it would have been if said tissue did not have said dysplastic epidermal lesions and/or a dermatological pre-cancerous disease

18. The method of claim 17, wherein the microwave energy is selected such as to stimulate a localised immune response at the dysplastic epidermal lesion and/or dermatological pre-cancerous disease and the administering of the therapeutically effective amount or dose of microwave energy to the dysplastic epidermal lesion and/or a dermatological pre-cancerous disease comprises repeatedly applying the microwave energy in a pulsed manner thus providing repeated rounds of localised hyperthermia at the dysplastic epidermal lesions and/or a dermatological pre-cancerous disease and repeated localised stimulation of the immune response at the dysplastic epidermal lesions and/or a dermatological pre-cancerous disease.
